(19) United States
(12) Patent Application Publication
(10) Pub. No.: US 2010/0286521 A1
(43) Pub. Date: Nov. 11, 2010

(54) MULTI-MODAL MEDICAL SCANNING METHOD AND APPARATUS

(75) Inventor: Nicholas Betts, Thebarton (AU)

Correspondence Address: Kevin McNamara;Signostics, PO Box 736, Torrensville, SA, 5031, omitted

(73) Assignee: SIGNOSTICS LIMITED, Torrensville, SA (AU)

(21) Appl. No.: 12/745,255

(22) PCT Filed: Nov. 27, 2008
(86) PCT No.: PCT/AU2008/001747

§ 371 Date: May 28, 2010

(30) Foreign Application Priority Data
Nov. 28, 2007 (AU) 2007906494

(51) Int. Cl. A61B 8/14

(52) U.S. Cl. 600/441

(57) ABSTRACT

A hand held ultrasound scanning apparatus of a type able to perform multiple scan modes and a hand held display and processing unit able to receive and display ultrasound scanline data, having a control for initiation and termination of ultrasound scanning where further operation of the same control causes the scan mode in use by the apparatus to move progressively through the available scan modes. The scan modes are selected from B-mode, M-mode, Gated Doppler, Power Doppler, Pulsed Wave Doppler, Color Doppler, Duplex Doppler, and 3D volume imaging.

### TECHNICAL FIELD

The present invention relates to a method and an apparatus for low cost ultrasound scanning.

### BACKGROUND ART

The use of ultrasound scanning of patients for medical diagnostic purposes dates to the mid-20th century. An ultrasound transducer is used to project a beam of ultrasound energy into a patient. The same or another transducer detects the echoes returned from features within the body. These echoes, called a scanline, are then converted to a form suitable for recording or display.

When a series of scanlines, spaced angularly apart, are acquired rapidly and displayed on a display screen, the familiar B-mode sector scan is achieved.

This is an arc of a circle, wherein the brightness of each pixel of the display is proportional to the magnitude of the ultrasound echo received from the corresponding point in the body being imaged.

A method from the prior art for collecting the required series of scanlines which are spaced angularly apart was to provide a single transducer in a handpiece attached to a data processing and display unit by an articulated arm. The articulated arm included means at each joint for tracking the movement of the joint. Tracking the position of each joint allowed the position and orientation of the handpiece, and hence of the transducer, to be known at all times. An operator would place the handpiece against the body of a patient, and sweep the handpiece in an arc to obtain the required set of angularly spaced scanlines.

A further development of this method was to place a motor in the handpiece. This motor moved the transducer, relative to the handpiece. The transducer rotated about an axis parallel to the surface of the body to be scanned. Means were provided within the handpiece for accurately determining the position of the transducer relative to the handpiece as the transducer moved.

In use, an operator placed the handpiece against the body of a patient, and held the handpiece still. The rotating transducer was activated to cause the ultrasound beam emitted by the transducer to sweep out a sector of the body. All the scanlines obtained in a single sector sweep were displayed to form a static B-mode image.

Since the relative position of the transducer was now known, it was no longer necessary for the exact position and orientation of the handpiece to be known, and the articulated arm was no longer required.

A further method for collecting the required angularly separated scanlines came with the advent of transducer arrays consisting of a number of piezo-electric crystals where the transmitting pulse can be delayed in sequence to each crystal and thus effect an electronic means to steer the ultrasound beam. An operator uses the system in the same way as for the motor driven transducer. The steered beam sweeps out a sector to produce the static B-mode scan, without the need for a motor to move the transducer.

Significant operator skill is necessary to locate a feature to be imaged such as a kidney or other organ. Basic anatomy knowledge allows the transducer to be positioned at the point on a patient's body from where the internal organ is most likely to be imaged. However, there is significant variation in human anatomy, and this positioning may not be optimal for a particular patient. The image produced from the first scans taken must then be used to find the optimal location for the transducer. Since each sector scan is performed discretely, considerable knowledge of the appearance of body organs which the operator is not seeking to scan may be necessary in order to accurately determine what is being imaged and to move the transducer to image the required organ.

One solution to this is to provide so-called real time B-mode, in which a series of static B-mode scans is rapidly and continuously acquired. These are then displayed sequentially, at approximately the rate at which they are acquired, that is, in real time.

The rate of display of the static B-mode scans must be at least sufficiently rapid such that the eye of the operator does not detect the transitions between scans. Since each scan needs to be assembled from its constituent scanlines, processed and displayed, the computer power required for this process is significant.

The size of area which can be scanned is limited to that which can practically be swept as a sector from the position at which the transducer is located.

A further known mode of use of ultrasound is M-mode. This mode employs a single transducer, or a single element of an array transducer. Ultrasound pulses are produced by the transducer regularly in time. The resultant scanlines are displayed continuously, regularly separated in space on a display. Thus the display shows a plot of echo return against time.

If the transducer is kept stationary with respect to a moving feature, the movement of that feature can be visualised. In medical imaging, the moving feature may be, for example, a heart valve.

It is increasingly desirable for medical ultrasound to be available to a wider range of medical personnel, in a wider range of circumstances.

The prior art devices have significant drawbacks in terms of portability and/or cost which limits the scope for wider deployment of medical ultrasound scanning.

The motor driven transducer has the disadvantage that the motor and the associated moving parts decrease the reliability and increase maintenance requirements. The motor also adds cost, and more importantly, weight and bulk to the handpiece. The motor will also add significantly to the power consumption of the ultrasound scanning system.

Nearly all modern medical ultrasound systems use an array of ultrasonic crystals in the transducer. The early designs used at least 64 crystals, with modern designs sometimes using up to a thousand crystals or more.

However, the cost of producing transducers with arrays of crystals is high. There is also a high cost in providing the control and processing circuitry, with a separate channel being required for each crystal. The transducers are usually manually manufactured, with the channels requiring excellent channel to channel matching and low cross-talk. The power consumption for electronic systems is also high, and is generally proportional to the number of channels being simultaneously operational.

These problems make the use of such systems in hand held, battery powered applications infeasible.

### DISCLOSURE OF THE INVENTION

Hand held ultrasound devices bring the advantages of ultrasound to the point of care, in the primary clinician's hands. However, such small battery powered devices cannot incorporate the traditional user interface having numerous knobs and a keyboard, without losing the advantage of small size and light weight. Also, the devices are hand held, meaning that the clinician does not always have a free hand to manipulate a traditional interface. Operations, such as scan mode changes which must be performed often and quickly need to be controlled in an easy and intuitive manner. Re-use of control devices allows for a reduction in the number of separate controls to be provided.

In one form of this invention there is provided a hand held ultrasound scanning apparatus of a type able to implement a plurality of scan modes and a hand held display and processing unit adapted to receive and display ultrasound scanline data, the apparatus including an operate control member adapted to be operated by a user, operation of said operate control member causing initiation and termination of ultrasound scanning, further operation of said operate control member causing the scan mode in use by the apparatus to move progressively through the implemented scan modes.

In preference the implemented scan modes are selected from B-mode, M-mode, Gated Doppler, Power Doppler, Pulsed Wave Doppler, Color Doppler, Duplex Doppler, and 3D volume imaging.

In a preferred embodiment there is provided a hand held ultrasound scanning apparatus of a type having at least two scan modes including B-mode ultrasound scanning and M-mode ultrasound scanning and a hand held display and processing unit adapted to receive and display ultrasound scanline data, the apparatus including an operate control adapted to be operated by a user, operation of said operate control causing initiation and termination of ultrasound scanning, further operation of said operate control causing the scan mode to move progressively through the at least two modes.

The control should be one that is easily operated with one hand, preferably the same hand as is supporting part of the hand held apparatus. Preferably, the operate control is a button which is operated for a time period not exceeding a selected threshold time period in order to start and stop scans, with the further operation which serves to change scan modes being to depress said button for a time period exceeding a selected threshold time period.

An alternative operate control is a scrollwheel with a capability to be depressed. Depressing the scrollwheel for briefly serves to start and stop the scan operation, the further operation of depressing the scrollwheel for a time period exceeding a selected threshold time period being the manner in which the scan mode is changed.

In a further form the invention may be said to lie in a method of use of a hand held ultrasound scanning device of the type described previously to image a selected feature. This is done by setting the device to scan using M-mode modality, operating the operate control to begin scanning, moving the probe unit over a body to be scanned, and then observing M-mode data displayed on the display and processing unit to determine when the selected feature is returning ultrasound echoes, and hence the location with respect to the ultrasound transducer is known.

The operate control is then used to set the device to scan using B-mode modality, and a sector B-mode scan of the selected feature is performed.

In a further form the method may be performed by setting the apparatus to scan using B-mode modality, operating the operate control member to begin scanning and moving a probe unit over a body to be scanned while a observing B-mode display to locate a feature such as a fetal heart or a pelvic floor, then further operating the operate control member to set the device to scan using M-mode and performing a continuing M-mode scan of the selected feature.

Such use allows for example, detection of foetal heart beat at an early stage of gestation.

Other objects and advantages of the present invention will become apparent from the following description, taken in connection with the accompanying drawings, wherein, by way of illustration and example, an embodiment of the present invention is disclosed.

### BRIEF DESCRIPTION OF THE DRAWINGS

FIG. 1 illustrates a hand held ultrasound apparatus according to a preferred embodiment of the present invention.

FIG. 2 is a block diagram representation of the apparatus of FIG. 1.

FIG. 3 is a diagrammatic representation of the use of M-mode modality for image scanning using the device of FIG. 1.

FIG. 4 is a scan resulting from the use of the device of FIG. 1 using M-mode modality for image scanning.

### BEST MODE FOR CARRYING OUT THE INVENTION

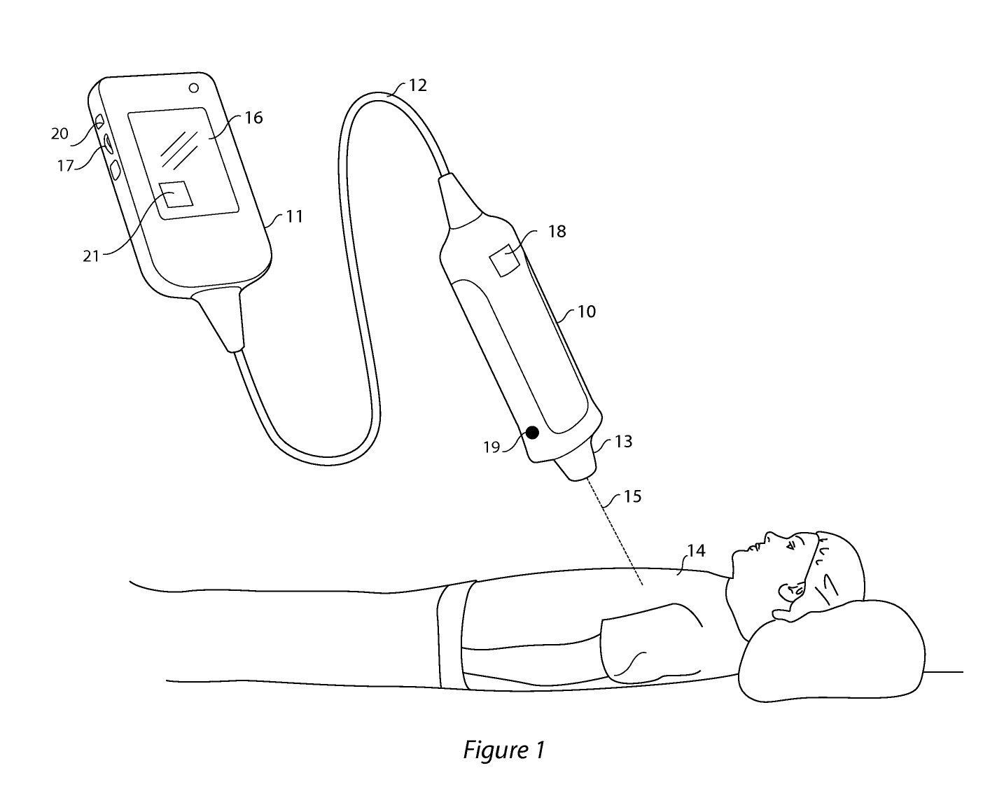

Referring now to FIG. 1, there is illustrated an ultrasound scanning system incorporating an embodiment of the invention. There is a hand held ultrasonic probe unit 10, a display and processing unit (DPU) 11 with a display screen 16 and a cable 12 connecting the probe unit to the DPU 11.

The probe unit 10 includes an ultrasonic transducer 13 adapted to transmit pulsed ultrasonic signals into a target body 14 and to receive returned echoes from the target body 14.

The transducer is adapted to transmit and receive in only a single direction at a fixed orientation to the probe unit, producing data for a single scanline 15. The system is a simple, low cost portable ultrasound scanning system. Additional transducers may be provided, at the expense of increased cost and complexity.

The probe unit further includes an orientation sensor 18 capable of sensing orientation or relative orientation about one or more axes of the probe unit. Thus, in general, the sensor is able to sense rotation about any or all of the axes of the probe unit.

The sensor may be implemented in any convenient form. In an embodiment the sensor consists of three orthogonally mounted gyroscopes. In further embodiments the sensor may consist of two gyroscopes, which would provide information about rotation about only two axes, or a single gyroscope providing information about rotation about only a single axis.

Since the distance between the mounting point of the sensor 18 and the tip of the transducer 13 is known, it would also be possible to implement the sensor with one, two or three accelerometers.

The orientation sensor may be any combination of gyroscopes and accelerometers mounted in relative position to one another so as to give information about the angular displacement of the probe unit.

In further embodiments, direction information for scanlines may be available for rotation about any or all axes of the probe unit.

Visual tracking systems using a camera to observe the movement of the probe and translate this into orientation tracking data could also be used. This has the disadvantage of requiring line of sight access to the probe unit at all times.

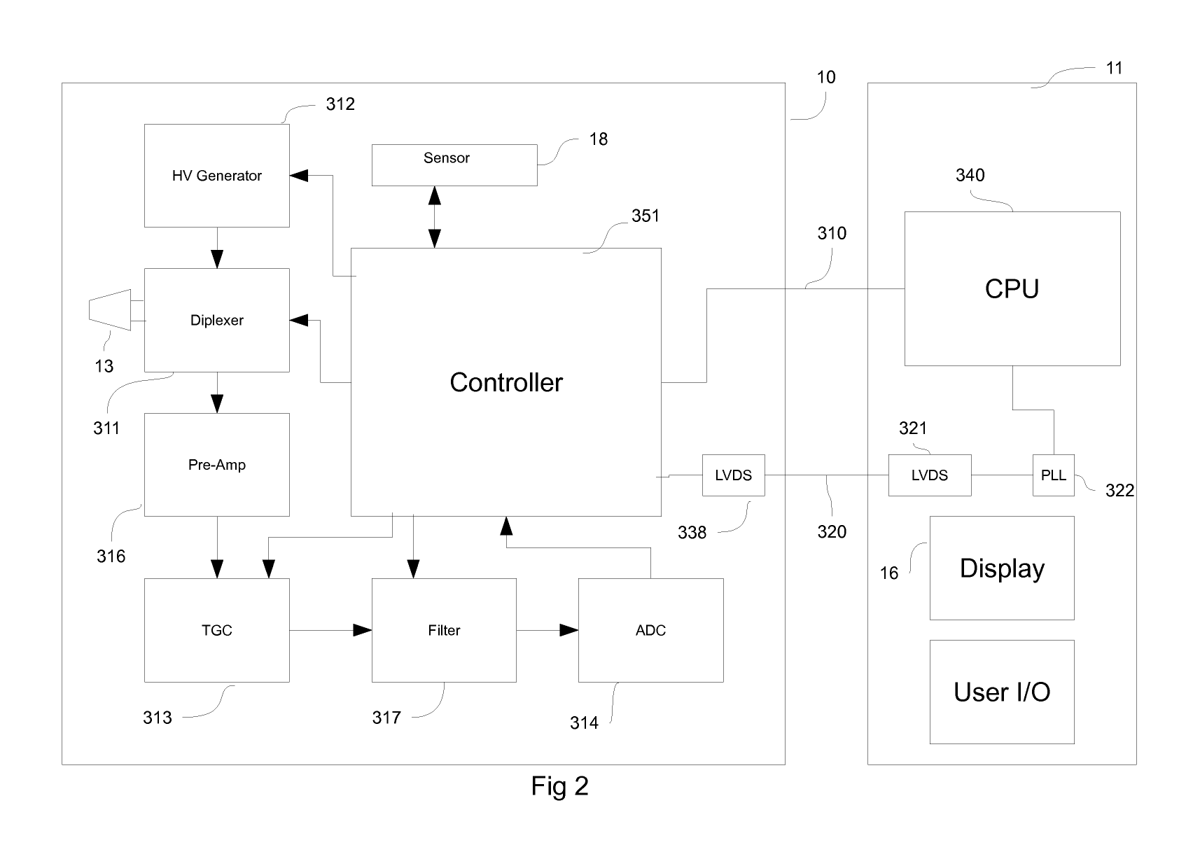

A block diagram of the ultrasonic scan system is shown in FIG. 2. There is a probe unit 10 and a DPU 11. The probe unit includes a controller 351 which controls all of the functions of the probe.

The DPU includes a main CPU 340.

The probe unit 10 communicates with the DPU 11 via a low speed message channel 310 and a high speed data channel 320. The message channel is a low power, always on connection.

The data channel is a higher speed and hence higher power consumption bus which is on only when required to transmit data from the probe unit to the DPU.

The probe unit includes a transducer 13 which acts to transmit and receive ultrasonic signals. A diplexer 311 is used to switch the transducer between transmit and receive circuitry.

On the transmit side the diplexer is connected to high voltage generator 312, which is controlled by controller 351 to provide a pulsed voltage to the transducer 13. The transducer produces an interrogatory ultrasonic pulse in response to each electrical pulse.

This interrogatory pulse travels into the body and is reflected from the features of the body to be imaged as an ultrasonic response signal. This response signal is received by the transducer and converted into an electrical received signal.

The depth from which the echo is received can be determined by the time delay between transmission and reception, with echoes from deeper features being received after a longer delay. Since the ultrasound signal attenuates in tissue, the signal from deeper features will be relatively weaker than that from shallower features.

The diplexer 311 connects the electrical receive signal to time gain compensation circuit (TGC) 313 via a pre-amp 316. The TGC applies amplification to the received signal. The characteristics of the amplification are selected to compensate for the depth attenuation, giving a compensated receive signal where the intensity is proportional to the reflectiveness of the feature which caused the echo. In general, the amplification characteristics may take any shape.

This compensated signal is passed to an analogue to digital converter (ADC) 314, via an anti-aliasing filter 317. The output of the ADC is a digital data stream representing the intensity of the received echoes over time for a single ultrasonic pulse.

There is an orientation sensor 18 which is adapted to provide information about angular rotation of the probe unit.

To perform a B-mode scan of a particular sector of a body to be imaged, a user applies the probe unit 10 to a body to be imaged 14. A scan is initiated by the user, by means of an operate control either on the probe unit or on the DPU. The activation of the control is detected by the controller 351 and communicated to the DPU 11 via the message channel 310.

The operate control may be in the form of a button 19 on the probe unit. It may also be in the form of a physical control located on the DPU 11 such as a depressible scrollwheel 17 which may be depressed to send an operate control signal. It may also be a “soft” control, in the form of a selectable icon 21 displayed on the screen of the DPU. In an embodiment where the screen is a touch screen, the icon may be directly selected by touch. In other embodiments, the icon may be selected by positioning a cursor using a cursor control such as a scrollwheel 17 which may be depressed to send a separate control signal, which, as in the illustrated embodiment may be the same as the scrollwheel used to send the operate signal. Context sensitive software may be used to determine the particular operating mode of the scrollwheel.

Any or all forms of the operate control, including alternative forms not illustrated, may be provided in a particular embodiment of the system.

The DPU responds with a message which includes any parameters which have been selected for the scan. The controller 351 controls the high voltage driver to produce the required pulse sequence to be applied via the diplexer to the transducer in order to perform a scan according to the parameters set by the user, or set as defaults in the DPU.

The user rotates the probe as required to sweep the ultrasound beam over the desired area, keeping linear displacement to a minimum, since this is a B-mode scan and it is desirable for all the scanlines to have a common origin.

In embodiments where rotation about all axes is not sensed, the user will also keep rotation about unsensed axes, that is, axes about which rotation is not detected by the sensor of the embodiment, to a minimum.

At the same time, data is received from the orientation sensor 18. This is the rotation about the sensed axes of the probe unit. It may be the angular change in the position of the probe unit since the immediately previous transducer pulse, or the orientation of the probe unit in some defined frame of reference. One such frame of reference may be defined by nominating one transducer pulse, normally the first of a scan sequence, as the zero of orientation.

The sensor data and the response signal are passed to the controller 351 where they are combined to give a scanline dataset. A scanline dataset comprises a sequential series of intensity values of the response signal combined with orientation information.

In the case of an M-mode scan, the sensor is disabled or data from it ignored. The user keeps the probe unit stationary at a location where the ultrasound beam intersects with a moving organ to be visualised.

The scanline dataset is generated in the controller 351. The scanline dataset is then passed to a protocol converter to be converted to a protocol suitable for transmission via the data channel. Any suitable protocol may be used. In this embodiment the protocol chosen for use on the data channel is 8b10b, which is well known in the art.

The 8b10b data is passed to an LVDS transmitter 338 and is transmitted via the data channel 320 to the DPU 11.

The LVDS data channel is received by the DPU via LVDS receiver 321 and phase locked loop 322. The 8b10b data is passed to the DPU processor 340. Protocol conversion is performed by processor 340 to recover the original scanline dataset.

An application is now run by the DPU processor 340 to process the scanline dataset for display on the display 16 of the DPU 11.

In the case of a B-mode scan, this display shows the scanlines in relation to each other according to the data from the orientation sensor. The display is thus a spatial representation of the real world features of the area being scanned.

In the case of an M-mode scan, sensor data is unavailable or ignored. The scanlines are displayed spread linearly across the display, with the spatial gap between the lines being proportional to the time interval between the reception of the lines. In the most common case, the time intervals will be constant, thus the lines will be evenly distributed across the display. In conventional M-mode, where the probe unit has been held stationary with respect to a moving anatomical feature, this will be a representation of the movement of that feature.

The high voltage generator 312 continues to provide the pulsed voltage to the transducer under control of the microcontroller and each pulse results in a scanline.

A B-mode scan is completed when the DPU determines from scanline orientation data that the required sector has been scanned or when the scan is terminated by user control.

An M-mode scan may be terminated by a user or by the expiry of a pre-set time.

Traditionally B-mode functionality has been used for sector scanning, and M-mode functionality has been used for imaging the motion of features. Skilled operators have employed expert knowledge of anatomy viewed with ultrasound to use sequential B-mode scans to home in on the organ to be scanned.

A hand held, low cost device such as the device of the invention makes the use of ultrasound devices for medical scanning feasible for a much wider range of medical professionals, such as general practitioner doctors, who may not have this level of knowledge. Further, the device may be used in situations where the user uses it relatively infrequently, so does not quickly build up this knowledge base.

In this case, finding a particular organ which it is desired to scan may be time consuming, using multiple manual B-mode scans.

However, true B-mode scans with spatial accuracy are only necessary when scanning the desired organ. Some spatial distortion is acceptable when the user is only seeking to position the transducer for a scan. An M-mode scan with a moving transducer will produce an image which is somewhat like a B-mode scan with a linear transducer array, where the depth of features is correctly shown, but the dimension parallel to the skin surface of such features will be shown incorrectly. We refer to this as a pseudo linear B scan.

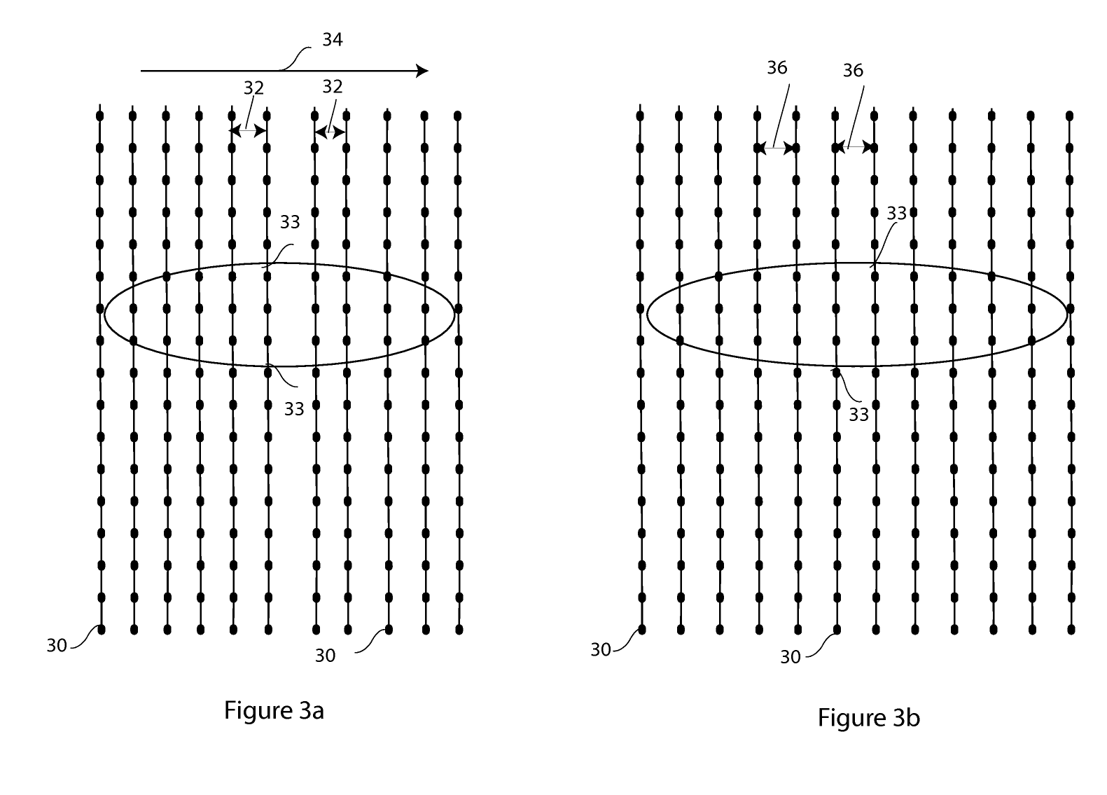

This is illustrated diagrammatically in FIG. 3. FIG. 3a shows a feature, in this case represented as an ellipse 31, which is isonified with a series of scanlines 30. These come from a transducer which is manually moved in the direction of the arrow 34. FIG. 3b shows the image 35 of the feature 31 on a display.

The distortion occurs because in the real world, the separation 32 between the points 33 at which each scanline 30 intersects a feature is determined by the speed at which the transducer is moved, and hence the distance which the transducer travels between the emission of each scanline, this emission being regular in time. As shown in FIG. 3b, the scanlines are displayed a regular distance 36 apart, with the distance being unrelated to the speed of movement of the transducer. This distorts the dimension parallel to the direction of movement of the transducer, while leaving the depth dimension correct.

In practice, the distortion is likely to be very much less than that shown in FIG. 3.

This distortion need not prevent a user from identifying major anatomical features, which may then be more accurately scanned using B-mode scanning as described previously.

Accordingly, a user wishing to perform a scan of a particular feature sets the device for M-mode scanning. The user then places the probe unit against the skin of a patient in approximately the correct position for scanning that feature. The user then activates scanning, and moves the probe unit, preferably in a reasonably uniform manner over the patient's skin. The probe unit transmits bursts of ultrasound energy into the body of the patient, and the resulting echoes from each burst are transmitted to the DPU as a scanline. No position or orientation data need be included in the scanline data.

The DPU displays each scanline as it is received, spacing the displayed scanlines uniformly across the display, and moving the display across the screen such that the displayed scanline are always the most recently received scanlines.

When the user identifies from the display that the desired feature is being imaged by the probe unit, the user halts the translational movement and changes to B-mode scanning in order to accurately image the required feature.

In a preferred embodiment, all of the start, stop and mode change instructions are conveyed to the device by multiple operations of the single operate control. The operate control may be in the form of a button 19 on the probe unit. It may also be in the form of a physical control located on the DPU 11 such as a depressible scrollwheel 17 which may be depressed to send an operate control signal. Additionally or alternatively, the control may be a button 20 located on the DPU.

It may also be a “soft” control, in the form of a selectable icon 21 displayed on the screen of the DPU. In an embodiment where the screen is a touch screen, the icon may be directly selected by touch. In other embodiments, the icon may be selected by positioning a cursor using a cursor control such as a scrollwheel 17 which may be depressed to send a separate control signal, which, as in the illustrated embodiment may be the same as the scrollwheel used to send the operate signal. Context sensitive software may be used to determine the particular operating mode of the scrollwheel.

In a preferred embodiment, the change from M-mode functionality to B-mode functionality, and vice-versa as required, is achieved by continued operation of the operate control. In embodiments where the operate control is depressed, this means that holding the control in a depressed state for longer than a selected threshold time period will initiate the mode change. Sequential operation of the control for less than this threshold period will start and stop the scan as appropriate, without changing the scanning mode.

In embodiments where more than two modes of operation are provided, the further continued operation of the operate control results in sequential selection of the available modes.

The pseudo linear B scan may also be directly useful in imaging features where the dimension parallel to the skin surface is irrelevant. This can arise in any circumstance where only the presence and/or the depth of a feature need be established.

An example of this is a scan for free fluid in the abdomen or thoracic cavity of a patient. Free fluid in such cases is a serious symptom, requiring prompt medical intervention. This may be surgical intervention or a procedure to puncture the skin to drain or withdraw the fluid. Thus it is necessary only to establish presence and location of the fluid in order to successfully diagnose and treat the condition.

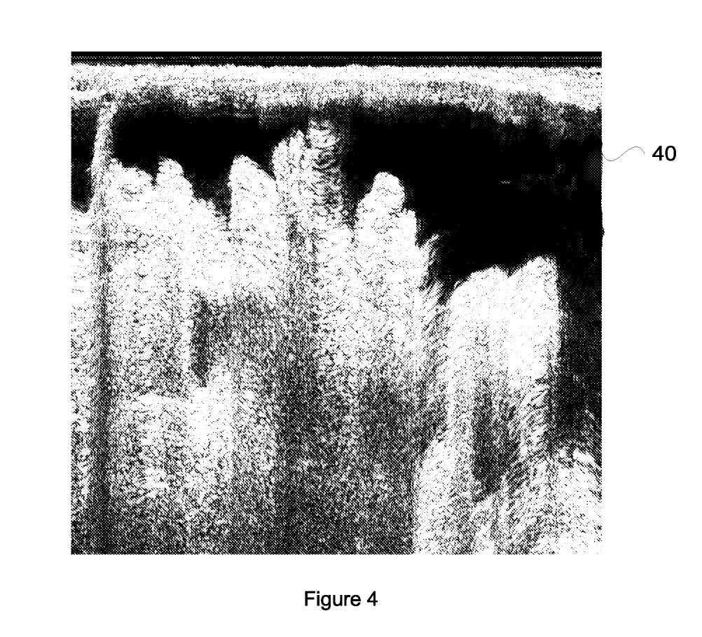

In order to scan for fluid in the abdominal cavity, a user sets the device to M-mode functionality, and operates the operate control to begin scanning, in this case by pressing and releasing the button 20. The user then moves the transducer over the patient's abdomen. A representation of a resultant scan in a human subject is shown in FIG. 4. The fluid is anechoic, and shows on the scan as the black region 40.

A similar technique can be used to check for the presence of a foreign body beneath the skin of a patient. Foreign bodies such as pieces of metal, wood or gravel may be lodged in soft tissue due to trauma. The ability to detect such bodies quickly and economically without the need for x-rays is a significant contribution to ensuring such bodies are removed during initial trauma treatment. In the case of radiolucent materials such as wood, ultrasound detection may be superior to x-ray detection.

In this case, M-mode scanning is used, moving the transducer laterally over a relatively large area where the presence of a foreign body is suspected. The acoustic appearance of foreign bodies in soft tissue is usually distinctive. When this distinctive signature is detected, lateral movement is stopped, B-mode scanning mode is entered, and a B-mode sector scan is undertaken to determine the size and shape and location of the foreign body. The ability to start and stop scanning and to move between M-mode and B-mode scanning by the operation of a single control makes this a quick and intuitive process, saving time, and lessening the possibility that the location discovered using M-mode searching will be inadvertently lost whilst changing scan mode.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1-13. (canceled)

14. A hand held ultrasound scanning apparatus of a type able to implement a plurality of scan modes and a hand held display and processing unit adapted to receive and display ultrasound scanline data, the apparatus including an operate control member adapted to be operated by a user, operation of said operate control member causing initiation and termination of ultrasound scanning, further operation of said operate control member causing the scan mode in use by the apparatus to move progressively through the implemented scan modes.

15. The apparatus of claim 14 wherein the implemented scan modes are selected from B-mode, M-mode, Gated Doppler, Power Doppler, Pulsed Wave Doppler, Color Doppler, Duplex Doppler, and 3D volume imaging.

16. The apparatus of claim 14 wherein the implemented scan modes are B-mode ultrasound scanning and M-mode ultrasound scanning.

17. The apparatus of claim 14 including a probe unit in data communication with the display and processing unit, said probe unit including an ultrasound transducer adapted to transmit and receive ultrasound signals and a processor adapted to receive and process signals from the transducer and to transmit data to the display and processing unit wherein the operate control member is located on the probe unit.

18. The apparatus of claim 14 wherein the control member is located on the display and processing unit.

19. The apparatus of claim 14 wherein the operate control member is a button, the operation of which is to depress said button for a time period not exceeding a selected threshold time period, the further operation being to depress said button for a time period exceeding a selected threshold time period.

20. The apparatus of claim 14 wherein the operate control member is a scrollwheel with a capability to be depressed the operation of which is to depress said scrollwheel for a time period not exceeding a selected threshold time period, the further operation being to depress said scrollwheel for a time period exceeding a selected threshold time period.

21. The apparatus of claim 14 wherein the operate control member is a scrollwheel with a capability to be depressed the operation of which is to depress said scrollwheel for a time period not exceeding a selected threshold time period, the further operation being to rotate said scrollwheel.

22. A method of use of the hand held ultrasound scanning apparatus of claim 14 to image a selected feature including the steps of
setting the apparatus to scan using M-mode modality,
operating the operate control member to begin scanning,
moving a probe unit over a body to be scanned,
observing M-mode data displayed to determine when the selected feature is returning ultrasound echoes,
further operating the operate control member to set the device to scan using B-mode modality,
performing a sector B-mode scan of the selected feature.

23. The method of claim 22 wherein the selected feature is a foreign body lodged in living tissue.

24. The method of claim 22 wherein the selected feature is a part of a needle or other medical device adapted to be inserted into a blood vessel of a patient.

25. A method of use of the hand held ultrasound scanning apparatus of claim 14 to image a selected feature including the steps of
setting the apparatus to scan using B-mode modality,
operating the operate control member to begin scanning,
moving a probe unit over a body to be scanned,
observing B-mode data displayed to determine when the selected feature is returning ultrasound echoes,
further operating the operate control member to set the device to scan using M-mode modality,
performing a continuing M-mode scan of the selected feature.

26. A hand held ultrasound scanning apparatus of a type having the capacity be used to perform ultrasound scanning in a plurality of modalities including an operate control member adapted to be operated by a user, operation of said control causing initiation and termination of ultrasound scanning, further operation of said control causing the scan mode of the device to move sequentially through the plurality of modalities.

27. The apparatus of claim 26 including a probe unit in data communication with the display and processing unit wherein said probe unit includes an ultrasound transducer adapted to transmit and receive ultrasound signals in a fixed direction relative to the probe unit.
